(19) United States
(12) Patent Application Publication
(10) Pub. No.: US 2025/0276194 A1
(43) Pub. Date: Sep. 4, 2025

(54) WEARABLE PHOTOTHERAPY APPARATUS

(71) Applicant: Thera B Medical Products, Inc., Hudsonville, MI (US)

(72) Inventor: Alexa Elizabeth Jones, South Amboy, NJ (US)

(21) Appl. No.: 19/066,128

(22) Filed: Feb. 27, 2025

Related U.S. Application Data
(63) Continuation of application No. 29/937,241, filed on Apr. 12, 2024.
(60) Provisional application No. 63/559,725, filed on Feb. 29, 2024.

(51) Int. Cl. A61N 5/06

(52) U.S. Cl. CPC A61N 5/0621; A61N 2005/0626; A61N 2005/0632; A61N 2005/0645

(57) ABSTRACT

A wearable phototherapy system including a swaddle assembly, a light mat and a controller. The swaddle assembly is configured to envelope an infant. The light mat is attachable to the swaddle assembly. The controller is electrically coupled to the light mat. Methods of use are likewise disclosed.

### CROSS-REFERENCE TO RELATED APPLICATION

The present application claims priority from U.S. Prov. Pat. App. Ser. No. 63/559,725, filed Feb. 29, 2024, entitled “Wearable Phototherapy Apparatus”, the entire specification of which is hereby incorporated by reference in its entirety.

The present application is a continuation of U.S. Des. patent application Ser. No. 29/937,241, filed Apr. 12, 2024, entitled “Swaddle For A Phototherapy Apparatus and Phototherapy Device”, the entire specification of which is hereby incorporated by reference in its entirety.

### BACKGROUND OF THE DISCLOSURE

### 1. Field of the Disclosure

The disclosure relates in general to medical devices, and more particularly, to a wearable phototherapy apparatus that is configured for use in the treatment of infant hyperbilirubinemia, also known as neonatal jaundice.

### 2. Background Art

Neonatal jaundice is a common condition in newborns characterized by the yellowing of the skin and eyes due to elevated levels of bilirubin, a yellow pigment produced during the breakdown of red blood cells. In newborns, jaundice typically appears within the first few days of life and is often harmless. However, in some cases, especially when bilirubin levels become excessively high, it can lead to complications such as brain damage, known as kernicterus.

The treatment for neonatal jaundice depends on various factors including the underlying cause, the infant's age, and the severity of jaundice. The most common approach is phototherapy, which involves exposing the skin of the infant skin to blue light at a particular range of wavelengths that help break down bilirubin into a form that can be more easily excreted by the body.

A number of different devices providing phototherapy have been developed. There nevertheless remains room for improvement. Amongst other areas where improvement is improvement in the application of the phototherapy, improvement in the systems that apply phototherapy, an improvement in the control of phototherapy and an improvement to the comfort of the infant.

### SUMMARY OF THE DISCLOSURE

The disclosure, in an aspect, is directed to a wearable phototherapy system for an infant. The system comprises a swaddle assembly and a light mat. The swaddle assembly comprises a body member, a pocket panel and an upper sealing member. The body member is adapted to encircle the infant from a neck region downward, defining a central spine region, a first side wing on a first side thereof and a second side wing on a side opposite the first wing. The pocket panel is positioned on an inner surface of the body member overlying at least a portion of the central spine region, and defining a cavity with an opening facilitating entry into the cavity. The pocket panel is light permeable. The upper sealing member includes a first side and a second side opposite the first side extending outwardly from a top end of the body member. The first and second sides cooperate to form a central slot adapted to encircle the neck of an infant when the body member encircles the infant.

The light mat comprises a flexible PCB assembly and an upper silicone layer. The flexible PCB assembly has a plurality light elements organized in a spaced apart manner along the flexible PCB assembly. The upper silicone layer overlies the flexible PCB assembly. The light mat is insertable through the opening into the cavity defined by the pocket panel and the body member, with plurality of light elements when energized oriented to direct light through the upper silicone layer and the pocket panel.

In some configurations, the first side and the second side are adapted to overlie a portion of an outer surface of the body member when encircling the infant, and are releasably attachable to an outer surface of the body member.

In some configurations, at least one of the first side wing and the second side wing are adapted to overlie the other of the first side wing and the second wing when encircling the infant, and wherein the first side wing and the second side wing are releasably attachable to each other.

In some configurations, the pocket panel extends toward the first side wing a distance beyond the first side of the upper sealing member and wherein the pocket panel extends toward a second wing side a distance beyond the second side of the upper sealing member, so as to be adapted to at least partially encircle the infant.

In some configurations, the swaddle assembly further includes a lower sealing member extending from a bottom end of the body member opposite the upper sealing member.

In some configurations, the lower sealing member is releasably attachable to an outer surface of the upper sealing member when encircling an infant so as to cover seal a lower end of the swaddle assembly.

In some configurations, the lower sealing member includes a plurality of openings extending therethrough.

In some configurations, the pocket panel of the body member comprises a mesh member that is one of transparent and translucent.

In some configurations, the system further includes a controller which is electrically coupled to the light mat and adapted to selectively energize the light elements of the light mat.

In some configurations, the controller is wired to the light mat about a lower end thereof.

In some configurations, the light mat further includes a lower silicone layer, wherein the upper silicone layer and the lower silicone layer cooperatively envelope the flexible PCB sheet therebetween.

In some configurations, the upper silicone layer includes a cavity aligning with each of at least a plurality of the light emitters, so as to overlay the same.

In some configurations, the light mat further defines an upper body region and a lower body region. Both regions are spaced apart from each other with a connector portion extending therebetween. The connector portion is narrower than each of the upper body region and the lower body region.

In some configurations, the upper body region defines a central portion coupled to the connector portion, a first upper wing on one side thereof and a second upper wing on an opposite side thereof.

In some configurations, the lower body region defines a central portion coupled to the connector portion, a first lower wing on one side thereof and a second lower wing on an opposite side thereof.

In some configurations, the controller is attached to a lower end of the central portion of the lower body region.

In another aspect of the disclosure, the disclosure is directed to a light mat for a wearable phototherapy system. The light mat comprises a lower silicone layer, an upper silicone layer and a flexible PCB sheet. The upper silicone layer is opposite the lower silicone layer. The flexible PCB sheet has a plurality of light emitters disposed on the flexible PCB sheet. The flexible PCB sheet is enveloped between the upper silicone layer and the lower silicone layer, with the plurality of light emitters positioned so as to emit light through the upper silicone layer.

In some configurations, the light mat further defines an upper body region and a lower body region. Both regions are spaced apart from each other with a connector portion extending therebetween. The connector portion is narrower than each of the upper body region and the lower body region.

In some configurations, the upper body region defines a central portion coupled to the connector portion, a first upper wing on one side thereof and a second upper wing on an opposite side thereof. The lower body region defines a central portion coupled to the connector portion, a first lower wing on one side thereof and a second lower wing on an opposite side thereof. The controller is attached to a lower end of the central portion of the lower body region.

In another aspect of the disclosure, the disclosure is directed to a method of treating an infant with the wearable phototherapy system disclosed herein. The method comprises the steps of providing the wearable phototherapy system; positioning a back of an infant on the central spine region of the body member; wrapping the first side wing over the infant; wrapping the second side wing over the infant; securing the first side wing and the second side wing together; extending the upper sealing member around a neck of the infant; securing the first side of the upper sealing member to an outer surface of the body member; sealing the second side of the upper sealing member to the outer surface of the body member; and energizing the light emitters of the light mat.

In some configurations, the method includes the steps of: providing a swaddle assembly, the swaddle assembly comprises a body member and a pocket panel and an upper sealing member. The body member is adapted to encircle the infant from a neck region downward, defining a central spine region, a first side wing on a first side thereof and a second side wing on a side opposite the first wing. The pocket panel is positioned on an inner surface of the body member overlying at least a portion of the central spine region, defining a cavity with an opening facilitating entry into the cavity, the pocket panel being light permeable. The upper sealing member includes a first side and a second side opposite the first side extending outwardly from a top end of the body member. The first and second sides cooperate to form a central slot adapted to encircle the neck of an infant when the body member encircles the infant. The method further includes the steps of: providing a light mat comprising a flexible PCB assembly having a plurality light elements organized in a spaced apart manner along the flexible PCB assembly; inserting the light mat into the cavity through the opening so that the light emitters of the Flexible PCB sheet face the pocket panel; wrapping an infant with the swaddle assembly so as to sandwich the Light mat between a back of an infant and the swaddle assembly; energizing the light emitters of the light mat.

In some configurations, the step of wrapping further includes the steps of: placing a back of the infant on the central spine region; wrapping the first side wing over the infant; wrapping the second side wing over the infant and over a portion of the first side wing; releasably attaching the second side wing and the second first side wing together; wrapping the upper sealing member about a neck of the infant, with the first side extending on one side of the neck and the second side extending on a second side of the neck; releasably attaching the first side of the upper sealing member to at least one of the first side wing and the second side wing; and releasably attaching the second side of the upper sealing member to at least one of the first side wing and the second side wing.

In some configurations, the swaddle assembly further includes a lower sealing member extending from the central spine region proximate the bottom end of the body member. The step of wrapping, in such configurations, further includes the step of: wrapping the lower sealing member about a lower end of the infant to overlie the body member; and releasably attaching the lower sealing member to at least one of the first side wing and the second side wing.

In some configurations, the method further includes the step of attaching the light mat to a controller.

In yet another aspect of the disclosure, the disclosure is directed to a wearable phototherapy system for an infant. The system includes a swaddle assembly, a light mat and a controller. The swaddle assembly comprises a body member a pocket panel, an upper sealing member and a lower sealing member. The body member is adapted to encircle the infant from a neck region downward, defining a central spine region, a first side wing on a first side thereof and a second side wing on a side opposite the first wing. The pocket panel is positioned on an inner surface of the body member overlying at least a portion of the central spine region, defining a cavity with an opening facilitating entry into the cavity, the pocket panel being light permeable. The upper sealing member includes a first side and a second side opposite the first side extending outwardly from a top end of the body member. The first and second sides cooperate to form a central slot adapted to encircle the neck of an infant when the body member encircles the infant. The lower sealing member is attached to the bottom end of the body member proximate the central spine region.

The light mat includes a flexible PCB assembly, an upper silicone layer and a lower silicone layer. The flexible PCB assembly has a plurality light elements organized in a spaced apart manner along the flexible PCB assembly. The upper silicone layer overlying the flexible PCB assembly. The lower silicone layer underlying the flexible PCB assembly. The flexible PCB assembly is sandwiched between the upper silicone layer and the lower silicone layer. The controller is electrically attachable to the light mat. The light mat is insertable through the opening into the cavity defined by the pocket panel and the body member, with plurality of light elements when energized oriented to direct light through the upper silicone layer and the pocket panel.

In some configurations, the light emitter comprises a plurality of LED's.

In some configurations, the LED's emit blue light within a range of 459 nm and 480 nm.

In some configurations, a treatment level has an irradiance intensity level of ˜70 μW/cm²/nm.

In some configurations, the effective illumination area of the light mat is approximately 180 sq. in.

In yet another aspect of the disclosure, the disclosure is directed to a wearable phototherapy system for an infant. The system comprises a swaddle assembly, a light mat and a controller. The swaddle assembly has a body member and an upper sealing member. The body member is adapted to encircle the infant from a neck region downward, defining a central spine region, a first side wing on a first side thereof and a second side wing on a side opposite the first wing. The upper sealing member includes a first side and a second side opposite the first side extending outwardly from a top end of the body member. The first and second sides cooperate to form a central slot adapted to encircle the neck of an infant when the body member encircles the infant. The light mat includes a flexible PCB assembly and an upper silicone layer. The flexible PCB assembly has a plurality light elements organized in a spaced apart manner along the flexible PCB assembly. The upper silicone layer overlies the flexible PCB assembly. The controller is electrically attachable to the light mat. The light mat is positionable on the central spine region of the body member and wrappable with the body member about the infant with plurality of light elements when energized oriented to direct light through the upper silicone layer and toward at least a back of the infant.

In some configurations, the light mat has a hall effect sensor and the swaddle assembly may include a magnet. Upon proper positioning of the light mat, the magnet actuates the hall effect sensor of the swaddle assembly.

In some configurations, the light mat is releasably attachable to the body member.

### BRIEF DESCRIPTION OF THE DRAWINGS

The disclosure will now be described with reference to the drawings wherein:

FIG. 1 of the drawings is a top plan view of a configuration of the wearable phototherapy system of the present disclosure;

FIG. 2 of the drawings is an exploded view of a configuration of the wearable phototherapy system of the present disclosure;

FIG. 3 of the drawings is an exploded view of a configuration of the light mat of the present disclosure;

FIG. 4 of the drawings is a cross-sectional view of a configuration of the light mat of the present disclosure;

FIG. 5 of the drawings is a perspective view of an infant within the wearable phototherapy system of the present disclosure; and

FIG. 6 of the drawings is multiple views of a controller for use with the wearable phototherapy system of the present disclosure.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

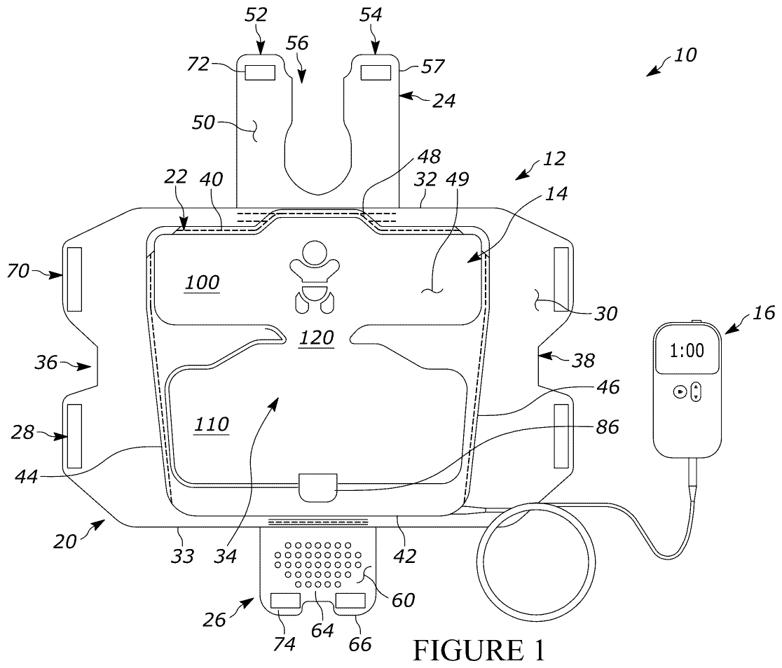

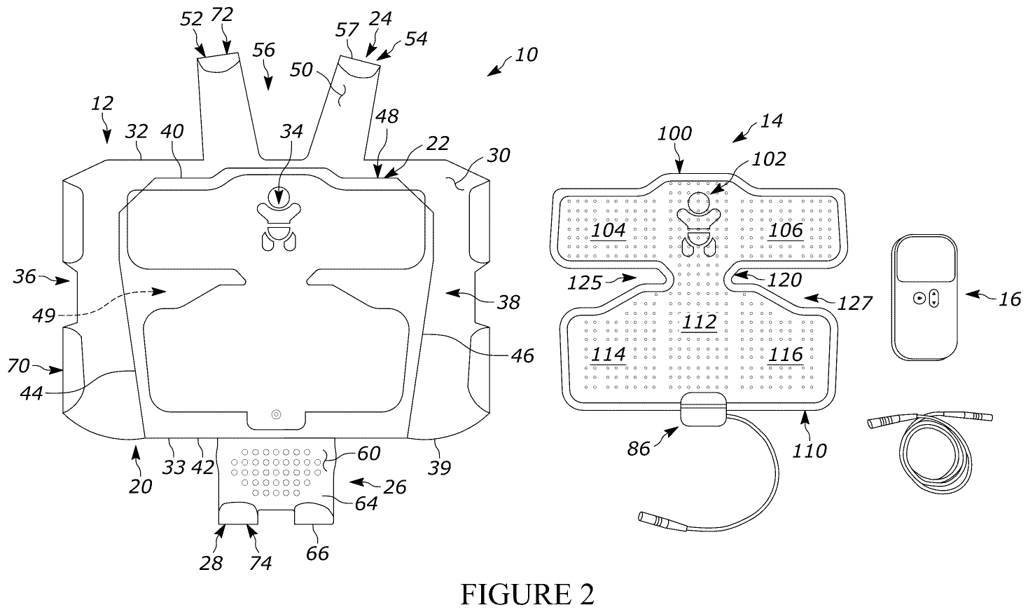

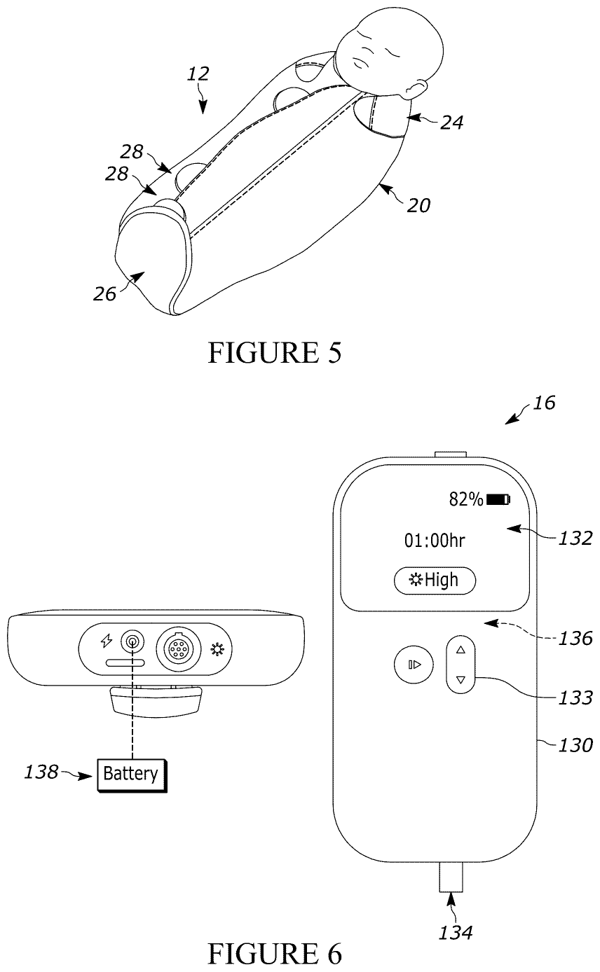

Referring now to the drawings and in particular to FIGS. 1 and 2, the wearable phototherapy system is shown generally at 10. The wearable phototherapy system is configured to be utilized by an infant for purposes of treatment of neonatal jaundice. With further reference to FIG. 5, it will be understood that the particular size and shape of the system is such that it is configured to fit an infant throughout the range of sizes of such infants. It will be understood that the system can be utilized for hospital use or home use.

With further reference to FIGS. 1 and 2, the wearable phototherapy system 10 is shown as comprising swaddle assembly 12, light mat 14 and controller 16. The swaddle assembly 12 is configured to enclose the body of an infant, generally below the neck. In the configuration shown, the swaddle assembly includes body member 20, pocket panel 22, upper sealing member 24, lower sealing member 26 and fasteners 28. The body member, the upper sealing member and the lower sealing member are formed from a two-way stretch material, such as Spunbond material. The material is capable of stretching in both a vertical direction and a horizontal direction. The material may be of the type that is typically used for disposable undergarments.

The body member 20 includes an inner surface 30, outer surface 31, top end 32 and bottom end 33. The inner surface is configured to be the portion that is abutting the infant, with the top end being proximate the head of the infant, and the bottom end being proximate the feet of the infant. The body member 20 includes a central spine region 34 with a first side wing 36 and a second side wing 38 opposite the first side wing 36. The body member 20 defines a perimeter 39 which is generally rectangular in a landscape orientation. It will be understood that the outer edges of the opposing first side wing 36 and second side wing 38 may have extensions or the like corresponding, for example to fasteners 28 (which will be described hereinbelow).

It will be understood that the central spine region 34 is generally centrally located and corresponds to the spine of the infant with the first side wing 36 encapsulating half of the body to one side and second side wing 38 encapsulating the other half of the body to the opposite side of the body.

Pocket panel 22 comprises a mesh member that is one of transparent and translucent. The pocket panel is light permeable. The pocket panel 22 includes top end 40, bottom end 42, first side 44 and second side 46. The pocket panel 22 overlies the inner surface 30 of the body member. The pocket panel 22 is coupled to the inner surface 30 of the body member along the bottom end 42 and the first and second sides 44, 46 to define a cavity 49. The two are not coupled across the top end 40 so as to define an opening 48 through which the light mat 14 and be inserted into the cavity 49. In the configuration shown, the material may comprise a Tulle mesh, while other materials are contemplated. Indeed, the materials are such that they are light permeable.

So as to provide for the proper positioning of the light mat, one of the pocket panel or the inner surface 30 of the body member 20 may include an image showing the proper placement of the light mat and/or the infant. In the configuration shown, the outline of the light mat is presented on one or both of the pocket panel and the body member, along with an image of an infant showing indicative of which direction the head should be positioned and which direction the legs should be positioned.

The upper sealing member 24 comprises a panel that extends from the top end 32 of the body member 20 proximate the central spine region 34. The upper sealing member includes inner surface 50 and outer surface 51, first side 52 and second side 54. The first side and the second side are spaced apart from each other so as to define a central slot therebetween. The slot is configured to receive the neck of the user with the first and second sides positioned on either side of the neck of the user. The upper sealing member can be formed from the same material as the body member 20.

Lower sealing member 26 is opposite the upper sealing member 24, and extends from the bottom end 33 of the body member also proximate the lower end of the central spine region 34. The lower sealing member 26 includes inner surface 60, outer surface 62, defining perimeter 66. A plurality of openings 64 extend through the lower sealing member 26, so as to permit circulation of air. The position and the number of openings that are utilized can be varied. In other configurations, the lower sealing member may omit openings.

Fasteners 28 comprises a plurality of hook and loop fasteners. In the configuration shown, the body member itself may be configured to comprise a loop portion of a hook and loop fastener, so that the fasteners comprise a hook portion of such fastener. The fasteners 28 include body member fasteners 70, that are positioned on the outer end of the first side wing 36 and on the outer end of the second side wing 38. The upper sealing member fasteners 72 comprise a pair of fasteners, one at the distal end of each of the first side 52 and the second side 54 of the upper sealing member. The lower sealing member fasteners comprise a pair of fasteners at a distal end of the lower sealing member. It will be understood that a greater number (or lesser number) of fasteners may be disposed on the body member, the upper sealing member and the lower sealing member. It will also be understood that in place of hook and loop type fasteners, other fasteners are contemplated for use, including, but not limited to adhesives, snaps, buttons, tabs and slots, interference fits, among others.

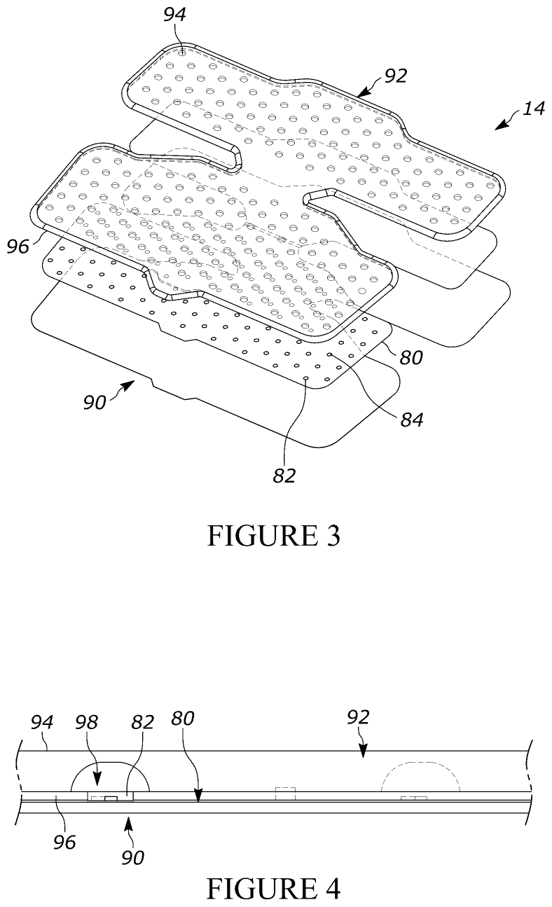

The light mat 14 is shown in FIGS. 3 and 4 as comprising a flexible PCB assembly 80, lower silicone layer 90 and upper silicone layer 92. The flexible PCB assembly comprises a plurality of light elements 82 (i.e., LED's) that are electrically coupled together in one of series and parallel connections (or through a plurality of independent circuits), a temperature sensor 84, and more preferably a plurality of temperature sensors, and a connector 86. The connector is in electrical communication with the light elements 82 and the temperature sensors 84. The connector 86 further includes a coupling that extends from the light mat for electrical coupling to the controller 16.

The flexible PCB assembly is sandwiched between the lower silicone layer 90 and the upper silicone layer 92. The upper silicone layer includes an upper surface which is substantially planar, and a lower surface which overlies and abuts the upper surface of the PCB assembly 80. A plurality of cavities, such as cavity 98 are defined into the lower surface 56 and correspond to the light elements, such that the light elements extend into or abut the cavities 98.

The upper and lower silicone layers are adhered to the flexible PCB assembly such that the entirety of the component is water resistant. For example, it is contemplated that a rating of IP67 can be achieved. Of course other levels of water resistance and/or dust resistance are contemplated. Additionally, the flexible PCB assembly may include printing on the upper surface thereof that provides visual indication as to the proper orientation of an infant relative to the light mat, as well as other instructions, warnings or the like.

In the configuration shown, the LED's emit blue light a peak wavelength of 470 nm (and preferably within a range of 459 nm and 480 nm). The treatment level is preferably at an irradiance intensity level of ˜70 μW/cm²/nm. Additionally, the effective illumination area of the light mat is approximately 180 sq. in. It is contemplated that the illumination area may be larger or smaller.

With further reference to FIGS. 1 and 2, the light mat 14 is configured so as to be positionable within cavity 49 formed by the interaction of the pocket panel 22 and the body member 20. To ensure proper coupling and positioning, the flexible PCB assembly may include a hall effect sensor (or other sensor) that senses whether or not the light mat has been properly positioned within the swaddle assembly. This can be achieved through the placement of a magnet on the body member positioned such that the hall effect sensor is triggered when the light mat is properly positioned within the cavity of the pocket panel. It will be understood that the light mat is inoperable until the sensor determines the proper positioning of the light mat within the swaddle assembly.

In the configuration shown, the light mat includes an upper body region 100, a lower body region 110 and a connector portion 120. The upper body region includes central portion 102, a first upper wing 104 and a second upper wing 106. The central portion may include an upward protrusion wherein otherwise the upper body region defines a generally rectangular configuration.

The lower body region includes a central portion 112, a first lower wing 114 and a second lower wing 116. The central portion may include a raised central region in an otherwise generally trapezoidal configuration.

The upper body region and the lower body region are aligned about their central portions 102, 112, with connector portion 120 extending therebetween. It will be understood that the connector portion and the central portion generally align with the central spine region of the body member, and the spine of the infant, when in use. Advantageously, the upper body region and the lower body region, about the wings thereof are configured to allow for independent movement, and so as to better follow the contours of the infant, to improve the direction of light from the light elements onto the body. For example, when in a flat configuration, a pair of opposing voids are formed on either side of the connector portion between the upper body region and the lower body region.

It is further contemplated that the light mat and the swaddle assembly may include a releasable coupling (such as a snap, a magnetic link or otherwise) so that the two are coupled in operable engagement, so as to minimize the movement of one relative to the other.

With reference to FIGS. 1, 2 and 6, controller 16 is shown as being coupled to the connector 86 of the light mat 14. Controller 16 includes a computing device 136 that is microprocessor based and that is configured to control the operation of the light mat. The controller further includes a housing 130 having a display 132, a plurality of buttons 133 and a connector attachable to the connector 86 through suitable wiring. In the configuration shown, the controller further includes an power adapter coupling which facilitates the providing of power (batter and/or wall outlet) to the controller and, in turn, the light mat.

The display is configured to provide output as to a number of parameters. The buttons may include an up and down button as well as a start/stop button. Other buttons are contemplated. It is contemplated that the display may comprise an OLED display. The connector may comprise an 8-pin medical grade connector. Of course, other connectors are contemplated.

In operation, the user is provided with the wearable phototherapy system 10 for an infant. It will be understood that the user may be provided with a single light mat 14 and controller 16, along with a plurality of swaddle assemblies 12. It is contemplated that the material from which the swaddle assemblies are made render the swaddle assemblies disposable. In other configurations, the swaddle assemblies may be washable or otherwise reusable.

Once provided, the user can attach the light mat 14 to the swaddle assembly 12. In particular, the light mat 14 can be inserted into the cavity 49 of the pocket panel so that the lower silicone layer overlies the inner surface of the body member 20 and the upper silicone layer is visible through the pocket panel.

When properly positioned, the hall effect sensor of the light mat can be actuated by the magnet of the swaddle assembly. In some configurations, the user may wish to releasably couple the light mat to the body member through the provided fastener. In other configurations, one or both of the foregoing may be eliminated.

It will further be understood that the swaddle assembly may include an outline, or instructions that facilitate the proper placement of the light mat within the pocket panel. For example, the body member or the pocket panel may include an outline that corresponds to the outline of the light mat so that the light mat can be inserted into the cavity of the pocket panel and the outer perimeter of the light mat can be aligned with the outline on the swaddle assembly.

Once assembled, the connector of the light mat can be attached to the controller to complete the assembly. It will be understood that the controller is itself attached to a power supply or a battery pack so as to be able to power both the controller and the light mat.

Once assembled, the swaddle assembly can be introduced to the infant. In particular, the infant is positioned back down onto the pocket panel and the body member so that the spine of the infant follows the central spine region of the body member of the swaddle and the central portion of the light mat. The top end of the body member of the swaddle should correspond to the neck of the infant, with the bottom end corresponding to the feet of the infant.

Once placed onto the swaddle assembly, the side wings are folded inwardly so as to encircle the infant. In particular, the first side wing can be extended over one side of the infant. The second side wing can be extended over the other side of the infant, and over the first side wing. The body member fasteners at the outer edge of the second side wing can engage the outer surface of the outer surface of the first side wing so as to releasably couple the two structures together.

Next, the upper sealing member can be placed in a coupling configuration. That is, the first and second sides are directed to overlie the outer surface of the body member, with the neck of the infant being captured by the central slot between the first and second sides. The upper sealing member fasteners couple the distal ends of the first and second sides to the outer surfaced of the body member.

Finally, the lower sealing member is moved into overlie the bottom end of the body member. The lower sealing member fasteners are configured to engage the outer surface of the body member in releasable engagement.

Once the body member, the upper sealing member and the lower sealing member are moved into the coupling configuration, the light mat is ready to be energized. Advantageously, and with reference to FIG. 5, due to the configuration of the swaddle assembly, the light mat is fully enveloped and the escape of light is minimized. It is believed that the light is captured within the swaddle assembly to such an extent that wearing of glasses or covering of the eyes of the infant is not necessary.

To energize the light mat, the user can, through the controller, choose the desired course of treatment. It is contemplated that the controller may have a number of different treatment protocols and options. Additionally, the controller can tabulate various data, including temperature of the light mat, the treatment times per session and total, among other parameters. Once the desired treatment is completed, the controller can automatically turn off the light mat. In other configurations, the user may manually energize and de-energize the light mat as desired.

It will be understood that the light mat may turn off automatically if certain conditions are sensed. For example, if a number of the LED's have malfunctioned, the light mat may be turned off by the controller, and the user may be alerted of a fault. Other fault conditions, such as excessively high temperatures may likewise cause the controller to de-energize the light mat. Still other conditions may require de-energizing of the light mat.

Once the treatment is completed, the infant can be removed from the system. Specifically, disengagement is achieved through the same steps executed in reverse. It will be understood that if the infant has soiled the swaddle assembly, the swaddle assembly may be disconnected from the light mat and discarded. In other configurations, it may be desirable to reuse the swaddle assembly. In such instances the swaddle assembly and the light mat may remain coupled. It will also be understood that the light mat may be decoupled from the controller as desired.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A wearable phototherapy system for an infant, comprising:
a swaddle assembly, the swaddle assembly comprising:
a body member adapted to encircle the infant from a neck region downward, defining a central spine region, a first side wing on a first side thereof and a second side wing on a side opposite the first wing;
a pocket panel positioned on an inner surface of the body member overlying at least a portion of the central spine region, defining a cavity with an opening facilitating entry into the cavity, the pocket panel being light permeable; and
an upper sealing member including a first side and a second side opposite the first side extending outwardly from a top end of the body member, the first and second sides cooperate to form a central slot adapted to encircle the neck of an infant when the body member encircles the infant;
a light mat comprising:
a flexible PCB assembly having a plurality light elements organized in a spaced apart manner along the flexible PCB assembly,
an upper silicone layer overlying the flexible PCB assembly;
wherein the light mat is insertable through the opening into the cavity defined by the pocket panel and the body member, with plurality of light elements when energized oriented to direct light through the upper silicone layer and the pocket panel.

2. The wearable phototherapy system of claim 1 wherein the first side and the second side are adapted to overlie a portion of an outer surface of the body member when encircling the infant, and are releasably attachable to an outer surface of the body member.

3. The wearable phototherapy system of claim 1 wherein at least one of the first side wing and the second side wing are adapted to overlie the other of the first side wing and the second wing when encircling the infant, and wherein the first side wing and the second side wing are releasably attachable to each other.

4. The wearable phototherapy system of claim 1 wherein the pocket panel extends toward the first side wing a distance beyond the first side of the upper sealing member and wherein the pocket panel extends toward a second wing side a distance beyond the second side of the upper sealing member, so as to be adapted to at least partially encircle the infant.

5. The wearable phototherapy system of claim 1 further comprising a lower sealing member extending from a bottom end of the body member opposite the upper sealing member.

6. The wearable phototherapy system of claim 5 wherein the lower sealing member is releasably attachable to an outer surface of the upper sealing member when encircling an infant so as to cover seal a lower end of the swaddle assembly.

7. The wearable phototherapy system of claim 6 wherein the lower sealing member includes a plurality of openings extending therethrough.

8. The wearable phototherapy system of claim 1 wherein the pocket panel of the body member comprises a mesh member that is one of transparent and translucent.

9. The wearable phototherapy system of claim 1 further comprising a controller, the controller being electrically coupled to the light mat and adapted to selectively energize the light elements of the light mat.

10. The wearable phototherapy system of claim 9 wherein the controller is wired to the light mat about a lower end thereof.

11. The wearable phototherapy system of claim 1 wherein the light mat further includes a lower silicone layer, wherein the upper silicone layer and the lower silicone layer cooperatively envelope the flexible PCB sheet therebetween.

12. The wearable phototherapy system of claim 11 wherein the upper silicone layer includes a cavity aligning with each of at least a plurality of the light emitters, so as to overlay the same.

13. The wearable phototherapy system of claim 1 wherein the light mat further defines an upper body region and a lower body region, both regions being spaced apart from each other with a connector portion extending therebetween, the connector portion being narrower than each of the upper body region and the lower body region.

14. The wearable phototherapy system of claim 13 wherein the upper body region defines a central portion coupled to the connector portion, a first upper wing on one side thereof and a second upper wing on an opposite side thereof.

15. The wearable phototherapy system of claim 14 wherein the lower body region defines a central portion coupled to the connector portion, a first lower wing on one side thereof and a second lower wing on an opposite side thereof.

16. The wearable phototherapy device of claim 15 wherein the controller is attached to a lower end of the central portion of the lower body region.

17. A light mat for a wearable phototherapy system comprising:
a lower silicone layer; and
an upper silicone layer opposite the lower silicone layer;
a flexible PCB sheet having a plurality of light emitters disposed on the flexible PCB sheet,
wherein the flexible PCB sheet is enveloped between the upper silicone layer and the lower silicone layer, with the plurality of light emitters positioned so as to emit light through the upper silicone layer.

18. The light mat of claim 16 wherein the light mat further defines an upper body region and a lower body region, both regions being spaced apart from each other with a connector portion extending therebetween, the connector portion being narrower than each of the upper body region and the lower body region.

19. The light mat of claim 18 wherein:
the upper body region defines a central portion coupled to the connector portion, a first upper wing on one side thereof and a second upper wing on an opposite side thereof;
the lower body region defines a central portion coupled to the connector portion, a first lower wing on one side thereof and a second lower wing on an opposite side thereof; and
wherein the controller is attached to a lower end of the central portion of the lower body region.

20. A method of treating an infant with the wearable phototherapy system of claim 1 comprising the steps of:
providing the wearable phototherapy system;
positioning a back of an infant on the central spine region of the body member;
wrapping the first side wing over the infant;
wrapping the second side wing over the infant;
securing the first side wing and the second side wing together;
extending the upper sealing member around a neck of the infant;
securing the first side of the upper sealing member to an outer surface of the body member;
sealing the second side of the upper sealing member to the outer surface of the body member; and
energizing the light emitters of the light mat.
